(19) United States
(12) Patent Application Publication
(10) Pub. No.: US 2026/0034057 A1
(43) Pub. Date: Feb. 5, 2026

(54) ORODISPERSIBLE TABLET, IN PARTICULAR FOR USE AS A FOOD SUPPLEMENT

(71) Applicant: GOETHE BIOTECHNOLOGY GMBH, Frankfurt am Main (DE)

(72) Inventors: Michael ULLRICH, Extertal (DE); Günther HARTENSTEIN, Landau (DE)

(73) Assignee: GOETHE BIOTECHNOLOGY GMBH, Frankfurt am Main (DE)

(21) Appl. No.: 19/099,754

(22) PCT Filed: Jul. 28, 2023
(86) PCT No.: PCT/EP2023/071022

§ 371 Date: Jan. 29, 2025

(30) Foreign Application Priority Data
Jul. 29, 2022 (DE) 10 2022 119 133.8

(51) Int. Cl. A61K 9/00; A61K 9/20; A61K 31/05; A61K 31/121; A61K 31/122; A61K 31/202; A61K 31/375; A61K 31/4045; A61K 31/7076; A61K 36/258; A61K 36/45; A61K 36/74; A61K 36/88

(52) U.S. Cl. CPC A61K 9/0056; A61K 9/2018; A61K 31/05; A61K 31/121; A61K 31/122; A61K 31/202; A61K 31/375; A61K 31/4045; A61K 31/7076; A61K 36/258; A61K 36/45; A61K 36/742; A61K 36/88

(57) ABSTRACT

An orodispersible tablet, in particular for use as a dietary supplement, comprising as main ingredients: an active agent, a disaccharide, and a phosphatidylcholine.

### CROSS-REFERENCE TO RELATED APPLICATION

This application is a national phase of International Application No. PCT/EP2023/071022, filed Jul. 28, 2023, which is hereby incorporated by reference in its entirety.

### FIELD

This disclosure relates to an orodispersible tablet and, in particular, to an orodispersible tablet for use as a dietary supplement.

### BACKGROUND

A large number of active ingredients for a variety of treatment purposes are known from natural medicine or natural healing. For example, the active ingredient cordycepin has been used for a long time in traditional Chinese medicine for a variety of treatment purposes. In particular, this active ingredient is said to be highly effective against various types of cancer or tumor diseases, both in terms of their development and their spread. It can be obtained from the medicinal mushroom Cordyceps, which is one of the most potent medicinal mushrooms used in traditional Chinese medicine. Athletes use cordyceps to improve their stamina and performance. People with a weakened immune system use cordyceps to strengthen their immune system, and those with autoimmune diseases use the regulating medicinal mushroom to curb it.

Cordycepin or 3′-deoxyadenosine is a derivative of the nucleoside adenosine, which differs from it in that the hydroxy group in the 3′-position is replaced by a hydrogen. It was originally obtained from the fungus Cordyceps militaris, but can now be produced synthetically. It has the chemical formula C₁₀H₁₃NsO₃ and a molar mass of 251.24 mol/g and the structure:

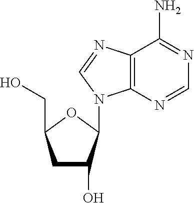

Furthermore, curcumin, as the biologically active ingredient in turmeric, is the subject of a large number of medical studies on many different disease patterns. Turmeric stimulates the production of gastric juice. As a spice, turmeric also has a positive influence on digestion; it can alleviate or prevent mild complaints such as bloating or a feeling of fullness after eating. In traditional Indonesian medicine, turmeric is used as the main ingredient in jamu, the traditional Indonesian remedies, to treat a variety of ailments, to strengthen the immune system and to prevent infections and respiratory diseases.

Cucurmin has the following structural formulas (top: keto form; bottom: enol form):

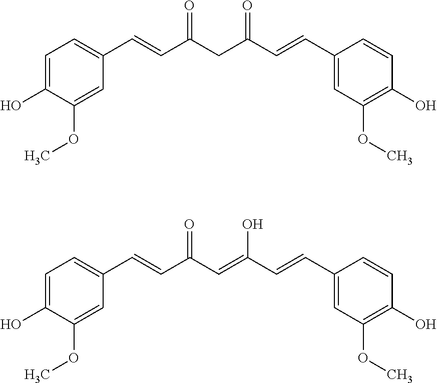 [SMILES: COc1cc(/C=C/C(=O)CC(=O)/C=C/c2ccc(O)c(OC)c2)ccc1O ; COc1cc(/C=C/C(=O)/C=C(O)/C=C/c2ccc(O)c(OC)c2)ccc1O]

Omega-3 fatty acids, hereinafter generally referred to as “omega-3”, are a subgroup within the omega-n fatty acids, which are unsaturated compounds. They were formerly commonly referred to as vitamin F. Omega-3 means that the last double bond in the polyunsaturated carbon chain of the fatty acid is present at the third-last C—C bond, counting from the carboxy end. Omega-3 is generally considered to be a valuable and health-promoting component of a daily diet. The fatty acids eicosapentaenoic acid (EPA) and docosahexaenoic acid (DHA) are considered to be particularly valuable and health-promoting.

### EPA and DPA Have the Structural Formula

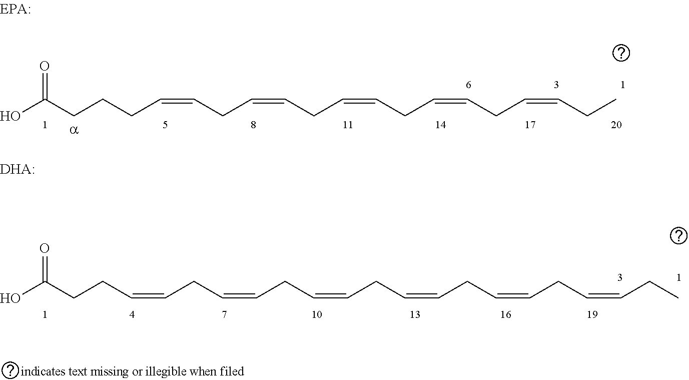 [SMILES: CC/C=C\C/C=C\C/C=C\C/C=C\C/C=C\CCCC(=O)O ; "DHA:" ; CC/C=C\C/C=C\C/C=C\C/C=C\C/C=C\C/C=C\CCC(=O)O]

Melatonin is a hormone produced by the pinealocytes in the pineal gland (epiphysis)—a part of the diencephalon—from serotonin. It regulates the day-night-rhythm of the human body and promotes sleep. It is a chemical compound from the group of tryptamines with 5-methoxyindole as a structural element.

Resveratrol is an Organic Compound with the Molecular Formula C₁₄H₁₂O₃ and the Structural Formula

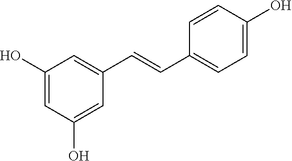 [SMILES: Oc1ccc(/C=C/c2cc(O)cc(O)c2)cc1]

from the group of polyphenols. It is a phytoalexin with antioxidant properties and is said to have numerous healing effects.

Vitamin C, also known as ascorbic acid, is a colorless, odorless, crystalline, water-soluble solid with a sour taste. Ascorbic acid is an organic acid, more precisely a vinylogous carboxylic acid; its salts are called ascorbates. Ascorbic acid exists in four different stereoisomeric forms, but only L-(+)-ascorbic acid exhibits biological activity. An important property in humans and some other species is its physiological effect as a vitamin. A deficiency can manifest itself in humans as scurvy. The name is therefore derived from the Latin name for the disease, scorbutus, with the negative prefix a-(un-, non-), thus the ‘antiscorbutic’ acid. Since ascorbic acid is easily oxidized, it acts as a redukton and is used as an antioxidant.

Ubiquinone-10 (also known as Q-10 or coenzyme Q10) is a quinone derivative with a lipophilic isoprenoid side chain, structurally related to vitamin K and vitamin E. The reduced, phenolic form is called ubiquinone or ubiquinol (QH2 for short). Ubiquinone-10 belongs to the ubiquinones. Q-10 is an electron and proton carrier between complex I or complex II and complex III of the respiratory chain and is used in cosmetic creams as well as in food supplements. It has the structural formula:

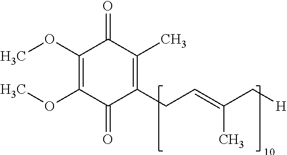

Due to their recognized effectiveness, there is a need for these active ingredients to be made more readily available and easier to use for simple, everyday use. In particular, it would be desirable to make them available in comparatively large quantities, which seems only partially possible given the scarcity and high price of these products.

### SUMMARY

This disclosure is therefore based on the task of specifying a dosage form that is particularly suitable for these purposes.

This task is solved in accordance with this disclosure by an orodispersible tablet comprising, as main components:
an active ingredient;
a disaccharide; and
a phosphatidylcholine.

Such an orodispersible tablet is particularly and especially intended for use as a dietary supplement.

### DETAILED DESCRIPTION

This disclosure is based on the consideration that, in view of the above-mentioned design objectives, the respective active ingredient should be administered in a particularly efficient manner so that the active ingredient can be supplied to the human organism in a particularly targeted manner. This makes it possible to use this scarce resource sparingly while maximizing the therapeutic potential. In order to make this possible, this disclosure provides for the active ingredient to be supplied in the form of an orodispersible tablet. Such an orodispersible tablet is intended to dissolve directly in the oral cavity after ingestion, whereby the active ingredient is released and can then be absorbed directly through the oral mucosa. This ensures that the active ingredient is delivered to the body's system in a highly efficient manner and bypasses the liver.

This also allows the so-called “first-pass effect” to be avoided. This describes the conversion of an active ingredient during its first passage (“first pass”) through the liver. The conversion (metabolization) that takes place during this process can result in an effective or ineffective metabolite.

The preferred active ingredients are cordycepin, curcumin, omega-3, melatonin, vitamin C, ubiquinone-10, resveratrol, saffron, blueberry, ginseng and/or a combination of ginseng with coffee, preferably individually or alternatively in combination with each other. Particularly preferred and considered to be independently inventive as an active ingredient is one or, in the case of fatty acids, eicosapentaenoic acid (EPA) and docosahexaenoic acid (DHA). In an alternative embodiment, considered to be independently inventive, barberry or so-called “cell disruptions” can also be provided as active ingredients. “Cell disruptions” are to be understood in particular as suitably prepared cell material, as could be provided, for example, in the context of a fresh cell therapy.

In order to be able to provide such an invention-based orodispersible tablet for the administration of one or more of the active ingredients mentioned, this active ingredient must be suitably incorporated into excipients or carriers that on the one hand, enable a tablet form that is stable in shape and has a certain shelf life and storage capacity, but which, on the other hand, can also be dissolved in the oral cavity so that the active ingredient is released and can be absorbed via the mucosa. To make this possible, it is planned to use a disaccharide and phosphatidylcholine, usually also referred to as lecithin, as further components for the orodispersible tablet in addition to the actual active ingredient.

In the preferred embodiment, trehalose is used as the disaccharide. Trehalose is a disaccharide consisting of two α,α′-1,1-glycosidically linked glucose molecules. Its systematic name is therefore 1-α-glucopyranosyl-1-α-glucopyranoside, in the short formula: Glc α(1→1)α Glc. Since both anomeric carbon atoms are involved in the O-glycosidic bond, trehalose belongs to the non-reducing sugars.

Phosphatidylcholine sunflower lecithin is provided as an alternative or additional beneficial further development. The combination of these additives, i.e. trehalose and lecithin, is particularly favorable for metabolism in the oral mucosa and is considered to be independently inventive.

In order to provide the aforementioned modes of action in a particularly reliable manner, the weight proportion of phosphatidylcholine is advantageously at least 50% higher than the weight proportion of the disaccharide, and is preferably at least twice as high.

In the manufacture of the composition intended as a raw material for the melt tablet, ultrasonic extraction is used to extract the respective active ingredients in a suitable manner, in accordance with an aspect regarded as independently inventive. After extraction, no filtration should be carried out, as this could be accompanied by a weakening of the active ingredients. Instead, according to one aspect of the invention, the extract is freed from solids contained therein exclusively by sedimentation (centrifugation).

During the extraction, according to one aspect of this disclosure, for example by means of a stirrer, a good distribution and homogenization of the active ingredient extract is ensured in order to be able to break it down better.

According to an aspect considered to be independently inventive, the production of the melt tablet involves the concept of lipoliophilization. This involves freeze-drying fats, which does not actually work under normal circumstances. To do this, the first step is to carefully stir a liquid or emulsion that is as homogeneous as possible, with all of the individual ingredients. This can be considered the most important step in the process of lipophilization. The finer the resulting homogeneous emulsion, the easier it is to carry out the process of lipophilization.

The excipient Trealose is then used, among other things, to build a scaffold for the fat molecules. This makes it possible to sublimate the water during the lipophilization process and to bind the fat molecules to the Trealose. The resulting liposome-Trealose mixture can be absorbed extremely well through the oral mucosa, as described.

The effect of the orodispersible tablet can be characterized as follows:

The above-mentioned additives enable lipophilic processing of the starting substances into an orodispersible tablet, which allows the active ingredient to be absorbed directly through the oral mucosa. The disaccharide, preferably trealose, provides a kind of supporting structure that enables the spatial form and stability of the orodispersible tablet. When it enters the oral cavity, however, the saliva enzymatically breaks down the disaccharide, so that the active ingredient is released for absorption through the oral mucosa.

The (sunflower) lecithin is also dissolved by the saliva (lipases) and absorbed through the oral mucosa.

The orodispersible tablet should be taken separately from meals and left in the mouth for at least 15 minutes without further food intake. During this period, complete resorption can take place in the mouth.

1. An orodispersible tablet for use as a food supplement, comprising:
an active agent;
a disaccharide; and
a phosphatidylcholine.

2. The orodispersible tablet according to claim 1, wherein the active agent is selected from the group consisting of cordycepin, curcumin, omega-3, melatonin, vitamin C, ubiquinone-10, resveratrol, saffron, blueberry, ginseng or a combination of ginseng with coffee.

3. The orodispersible tablet as claimed in claim 1, wherein the disaccharide is trehalose.

4. The orodispersible tablet as claimed in claim 1, wherein the phosphatidylcholine is sunflower lecithin.

5. The orodispersible tablet according to claim 1, wherein a proportion by weight of phosphatidylcholine is at least 50% higher than proportion by weight of phosphatidylcholine.

6. The orodispersible tablet according to claim 5, wherein the proportion by weight of phosphatidylcholine is at least twice the proportion by weight of phosphatidylcholine.

7. The orodispersible tablet as claimed in claim 2, wherein the disaccharide is trehalose.

8. The orodispersible tablet as claimed in claim 2, wherein the phosphatidylcholine is sunflower lecithin.

9. The orodispersible claimed in claim 3, wherein the phosphatidylcholine is sunflower lecithin.

10. The orodispersible tablet as claimed in claim 7, wherein the phosphatidylcholine is sunflower lecithin.

11. The orodispersible tablet according to claim 2, wherein a proportion by weight of phosphatidylcholine is at least 50% higher than proportion by weight of phosphatidylcholine.

12. The orodispersible tablet according to claim 3, wherein a proportion by weight of phosphatidylcholine is at least 50% higher than proportion by weight of phosphatidylcholine.

13. The orodispersible tablet according to claim 4, wherein a proportion by weight of phosphatidylcholine is at least 50% higher than proportion by weight of phosphatidylcholine.

14. The orodispersible tablet according to claim 11, wherein the proportion by weight of phosphatidylcholine is at least twice the proportion by weight of phosphatidylcholine.

15. The orodispersible tablet according to claim 12, wherein the proportion by weight of phosphatidylcholine is at least twice the proportion by weight of phosphatidylcholine.

16. The orodispersible tablet according to claim 13, wherein the proportion by weight of phosphatidylcholine is at least twice the proportion by weight of phosphatidylcholine.
